(19) United States
(12) Patent Application Publication
(10) Pub. No.: US 2024/0108314 A1
(43) Pub. Date: Apr. 4, 2024

(54) ULTRASONIC DIAGNOSTIC APPARATUS AND NON-TRANSITORY COMPUTER READABLE MEDIUM

(71) Applicant: Canon Medical Systems Corporation, Otawara-shi (JP)

(72) Inventor: Akio GODA, Otawara (JP)

(73) Assignee: Canon Medical Systems Corporation, Otawara-shi (JP)

(21) Appl. No.: 18/480,600

(22) Filed: Oct. 4, 2023

(30) Foreign Application Priority Data
Oct. 4, 2022 (JP) 2022-160416

(51) Int. Cl. A61B 8/08; G01S 7/52

(52) U.S. Cl. CPC A61B 8/488; G01S 7/52085

(57) ABSTRACT

According to one embodiment, an ultrasonic diagnostic apparatus includes processing circuitry. The processing circuitry sets a scan order such that among multiple ensemble groups each including multiple ultrasonic transmission-reception operations performed on a same scan line in each of partial scan regions of a scan region, a distance between temporally adjacent ultrasonic transmission-reception operations of different ensemble groups is not less than a first threshold, and a time difference between ultrasonic transmission-reception operations of the different ensemble groups with respect to spatially adjacent scan lines is not less than a second threshold. The processing circuitry causes ultrasonic waves to be transmitted and received according to the scan order.

### CROSS-REFERENCE TO RELATED APPLICATIONS

This application is based upon and claims the benefit of priority from Japanese Patent Application No. 2022-160416, filed Oct. 4, 2022, the entire contents of which are incorporated herein by reference.

### FIELD

Embodiments described herein relate generally to an ultrasonic diagnostic apparatus and a non-transitory computer readable medium.

### BACKGROUND

In a Doppler imaging method that estimates and images a displacement amount and a velocity of a biological tissue, such as a blood flow, a transmission-reception control method called interleave scanning is generally adopted. Interleave scanning is a method that does not continuously perform transmission and reception in the same beam position when collecting data columns in the same position, but groups multiple beam positions as one group and sequentially performs transmission and reception in the multiple beam positions included in the group. This enables extension of the sampling cycle for the same beam position and measurement of a low blood flow rate without a decrease in the frame rate.

In interleave scanning, however, when received beams are to be formed in adjacent beam positions, they are easily affected by the residual component in the immediately preceding beam position, and when the received beams are converted into an image, a residual multiplex noise in the form of a vertical stripe is generated. There is a method in which dummy transmission is performed according to the number of data pieces at the head of each interleave scanning in order to reduce the residual multiplex noise. This method, however, has a drawback in that the frame rate decreases due to an increase in the number of dummy transmissions along with an increase in the number of data pieces.

### BRIEF DESCRIPTION OF THE DRAWINGS

FIG. 1 is a block diagram showing an example of a configuration of an ultrasonic diagnostic apparatus according to an embodiment.

FIG. 2 is a flowchart for explaining an operation of the ultrasonic diagnostic apparatus according to the embodiment.

FIG. 3 is a diagram for explaining a scan region.

FIG. 4 is a diagram showing a first example of setting a scan order according to the embodiment.

FIG. 5 is a diagram for explaining a simplified ultrasonic scan chart.

FIG. 6 is a diagram showing a second example of setting a scan order according to the embodiment.

FIG. 7 is a diagram showing a third example of setting a scan order according to the embodiment.

FIG. 8 is a diagram showing a fourth example of setting a scan order according to the embodiment.

FIG. 9 is a diagram showing a fifth example of setting a scan order according to the embodiment.

FIG. 10 is a diagram showing a sixth example of setting a scan order according to the embodiment.

### DETAILED DESCRIPTION

In general, according to one embodiment, an ultrasonic diagnostic apparatus includes processing circuitry. The processing circuitry sets a scan order such that among multiple ensemble groups each including multiple ultrasonic transmission-reception operations performed on a same scan line in each of partial scan regions of a scan region, a distance between temporally adjacent ultrasonic transmission-reception operations of different ensemble groups is not less than a first threshold, and a time difference between ultrasonic transmission-reception operations of the different ensemble groups with respect to spatially adjacent scan lines is not less than a second threshold. The processing circuitry causes ultrasonic waves to be transmitted and received according to the scan order.

Hereinafter, embodiments of an ultrasonic diagnostic apparatus will be described in detail with reference to the accompanying drawings.

### First Embodiment

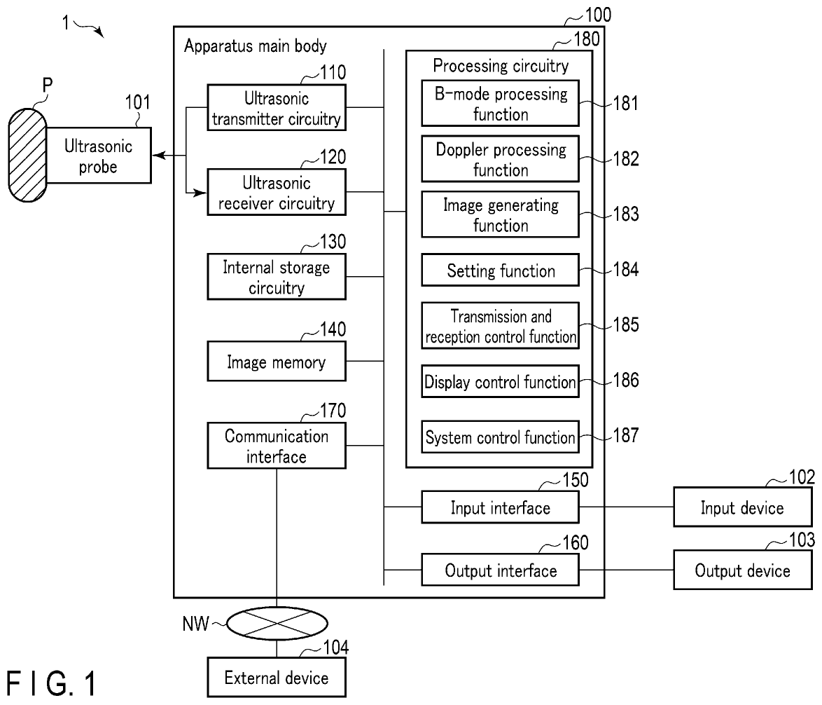

FIG. 1 is a diagram showing an example of a configuration of an ultrasonic diagnostic apparatus according to a first embodiment. The ultrasonic diagnostic apparatus 1 shown in FIG. 1 includes an apparatus main body 100 and an ultrasonic probe 101. The apparatus main body 100 is connected to an input device 102 and an output device 103. The apparatus main body 100 is also connected to an external device 104 via a network NW. The external device 104 is, for example, a server equipped with picture archiving and communication systems (PACS) and a workstation capable of executing post processing.

The ultrasonic probe 101 executes ultrasonic scanning of a scan region inside a living body P, which is a subject, under the control of, for example, the apparatus main body 100. The ultrasonic probe 101 includes, for example, an acoustic lens, one or more matching layers, a plurality of vibrators (piezoelectric elements), a backing material, etc. The acoustic lens is formed of, for example, silicone rubber, and converges ultrasonic beams. The one or more matching layers perform impedance matching between the plurality of transducers and the living body. The backing material prevents propagation of ultrasonic waves backward in a radial direction from the plurality of transducers. The ultrasonic probe 101 is, for example, a one-dimensional array linear probe in which a plurality of transducers are arranged along a predetermined direction. The ultrasonic probe 101 is detachably connected to the apparatus main body 100. The ultrasonic probe 101 may be provided with a button which is pressed when an offset process, an operation of freezing an ultrasonic image (i.e., freeze operation), etc., are performed.

The plurality of transducers generate ultrasonic waves based on a drive signal supplied from ultrasonic transmitter circuitry 110 (described later) that is included in the apparatus main body 100. Ultrasonic waves are thereby transmitted from the ultrasonic probe 101 to the living body P. When the ultrasonic waves are transmitted from the ultrasonic probe 101 to the living body P, the transmitted ultrasonic waves are sequentially reflected on an acoustic impedance discontinuous surface of a body tissue of the living body P, and are received as reflected wave signals by the plurality of piezoelectric transducers. The amplitude of the received reflected wave signals depends on a difference in acoustic impedance on the discontinuous surface to which the ultrasonic waves are reflected. If a transmitted ultrasonic pulse is reflected by bloodstream or a surface of the cardiac wall or the like that is in motion, the frequency of the reflected wave signals is shifted due to the Doppler effect according to the moving object's velocity component in the direction of ultrasonic transmission. The ultrasonic probe 101 receives the reflected wave signals from the living body P, and converts the reflected wave signals into electric signals.

FIG. 1 illustrates a connection relationship between a single ultrasonic probe 101 and the apparatus main body 100. However, a plurality of ultrasonic probes can be connected to the apparatus main body 100. Which of the connected ultrasonic probes is to be used for the ultrasonic scanning can be selected freely through, for example, a software button on a touch panel described later.

The apparatus main body 100 is an apparatus that generates an ultrasonic image based on the reflected wave signals (also referred to as “echo signals”) received by the ultrasonic probe 101. The apparatus main body 100 includes the ultrasonic transmitter circuitry 110, ultrasonic receiver circuitry 120, internal storage circuitry 130, an image memory 140, an input interface 150, an output interface 160, a communication interface 170, and processing circuitry 180.

The ultrasonic transmitter circuitry 110 is a processor that supplies a drive signal to the ultrasonic probe 101. The ultrasonic transmitter circuitry 110 is realized by, for example, a trigger generation circuit, a delay circuit, a pulser circuit, etc. The trigger generation circuit generates a rate pulse for forming ultrasonic waves for transmission repeatedly and at a predetermined rate frequency. The delay circuit gives a delay time for each piezoelectric element to each rate pulse generated by the trigger generation circuit. This delay time is needed to converge the ultrasonic waves generated from the ultrasonic probe into a beam and determine the transmission directivity. The pulser circuit applies a drive signal (drive pulse) to a plurality of ultrasonic vibrators provided in the ultrasonic probe 101 at the timing based on the rate pulse. The transmission direction from the surfaces of the piezoelectric vibrators can be freely adjusted by varying the delay time given to each rate pulse by the delay circuit.

The ultrasonic transmitter circuitry 110 can freely change the output intensity of the ultrasonic waves through the drive signal. In the ultrasonic diagnostic apparatus, an influence of the attenuation of the ultrasonic waves in the living body P can be reduced by increasing the output intensity. The ultrasonic diagnostic apparatus can acquire a reflected wave signal having a large S/N ratio at the time of reception by reducing the influence of the attenuation of the ultrasonic waves.

In general, when an ultrasonic wave is propagated inside the living body P, the strength of the vibration of the ultrasonic wave (also referred to as “acoustic power”) corresponding to the output intensity is attenuated. The attenuation of the acoustic power is caused by absorption, scattering, reflection, etc. The degree of attenuation of the acoustic power depends on the frequency of the ultrasonic waves and the distance of the ultrasonic waves in the radial direction. For example, the degree of attenuation is increased by increasing the frequency of the ultrasonic waves. Also, the degree of attenuation is increased as the distance of the ultrasonic wave in the radiation direction becomes longer.

The ultrasonic receiver circuitry 120 is a processor that performs various kinds of processing on the reflected wave signals received by the ultrasonic probe 101 and generates received signals. The ultrasonic receiver circuitry 120 generates received signals for the reflected wave signals of the ultrasonic waves obtained by the ultrasonic probe 101. Specifically, the ultrasonic receiver circuitry 120 is realized by, for example, a preamplifier, an A/D converter, a demodulator, a beamformer (adder), etc. The preamplifier performs gain correction by amplifying, for each channel, the reflected wave signals received by the ultrasonic probe 101. The A/D converter converts the gain-corrected reflected wave signals into digital signals. The demodulator demodulates the digital signals. The beam former, for example, gives the demodulated digital signals a delay time needed to determine the reception directivity, and adds a plurality of digital signals given the delay time. The addition processing performed by the beam former generates received signals in which a reflection component from the direction corresponding to the reception directivity is emphasized. The received signals may also be referred to as IQ signals. In addition, the ultrasonic receiver circuitry 120 may store the received signals (IQ signals) in the internal storage circuitry 130 (described later), or output the received signals (IQ signals) to the external device 104 via the communication interface 170.

The internal storage circuitry 130 includes, for example, a processor-readable storage medium, such as a magnetic storage medium, an optical storage medium, or a semiconductor memory. The internal storage circuitry 130 stores therein programs, various types of data and the like for implementing ultrasonic transmission and reception. The programs and various types of data may be pre-stored in, for example, the internal storage circuitry 130. Alternatively, the programs and various types of data may be, for example, stored and distributed in a non-transitory storage medium, and read from the non-transitory storage medium to be installed in the internal storage circuitry 130. In accordance with an operation that is input via the input interface 150, the internal storage circuitry 130 stores B-mode image data, contrast image data, image data relating to a blood flow image, and the like that are generated by the processing circuitry 180. The internal storage circuitry 130 can also transfer the stored image data to the external device 104 or the like via the communication interface 170. The internal storage circuitry 130 may store the received signals (IQ signals) generated by the ultrasonic receiver circuitry 120, or transfer the received signals (IQ signals) to the external device 104 or the like via the communication interface 170.

The internal storage circuitry 130 may be a drive device or the like which reads and writes various kinds of information to and from a portable storage medium such as a CD drive, a DVD drive, and a flash memory. The internal storage circuitry 130 can also write the stored data into a portable storage medium, and store the data in the external device 104 via the portable storage medium.

The image memory 140 includes, for example, a processor-readable storage medium, such as a magnetic storage medium, an optical storage medium, or a semiconductor memory. The image memory 140 stores image data corresponding to multiple frames immediately preceding a freeze operation that is input via the input interface 150. The image data stored in the image memory 140 is, for example, continuously displayed (cine-displayed).

The internal storage circuitry 130 and the image memory 140 need not necessarily be realized by independent storage devices. The internal storage circuitry 130 and the image memory 140 may be realized by a single storage device. The internal storage circuitry 130 and the image memory 140 may each be realized by a plurality of storage devices.

The input interface 150 receives various instructions from an operator via the input device 102. The input device 102 is, for example, a mouse, a keyboard, a panel switch, a slider switch, a trackball, a rotary encoder, an operation panel, or a touch command screen (TCS). The input interface 150 is connected to the processing circuitry 180, for example, via a bus, thereby converting an operational command that is input by the operator into an electric signal and outputting the electric signal to the processing circuitry 180. The input interface 150 is not limited to a component that is connected to physical operational components such as a mouse and a keyboard. Examples of the input interface also include circuitry that receives an electric signal corresponding to an operational command input from an external input device provided separately from the ultrasonic diagnostic apparatus 1 and outputs the electric signal to the processing circuitry 180.

The output interface 160 is, for example, an interface for outputting an electric signal from the processing circuitry 180 to the output device 103. The output device 103 is any display such as a liquid crystal display, an organic EL display, an LED display, a plasma display, or a CRT display. The output device 103 may be a touch-panel display that also serves as the input device 102. The output device 103 may further include a speaker that outputs voice in addition to a display. The output interface 160 is connected to the processing circuitry 180, for example, via a bus, and outputs the electric signal from the processing circuitry 180 to the output device 103.

The communication interface 170 is connected to the external device 104, for example, via the network NW to perform data communication with the external device 104.

The processing circuitry 180 is, for example, a processor acting as a nerve center of the ultrasonic diagnostic apparatus 1. The processing circuitry 180 executes a program stored in the internal storage circuitry 130, thereby implementing a function corresponding to the program. The processing circuitry 180 has, for example, a B-mode processing function 181, a Doppler processing function 182, an image generating function 183, a setting function 184, a transmission-reception control function 185, a display control function 186, and a system control function 187.

The B-mode processing function 181 is a function to generate B-mode data based on a received signal from the ultrasonic receiver circuitry 120. With the B-mode processing function 181, the processing circuitry 180 performs, for example, envelope detection processing, logarithmic compression processing, and the like on the signal received from the ultrasonic receiver circuitry 120 to generate data (B-mode data) that expresses a signal intensity by brightness. The generated B-mode data is stored in a RAW data memory (not shown) as B-mode RAW data on a two-dimensional ultrasonic scan line (raster).

The Doppler processing function 182 is a function to analyze the frequency of the signal received from the ultrasonic receiver circuitry 120 and thereby generate data (Doppler information) of an extraction of Doppler effect-based motion information of a moving object present in a region of interest (ROI) set in a scan region. The generated Doppler information is stored in a RAW data memory (not shown) as Doppler RAW data (also referred to as “Doppler data”) on a two-dimensional ultrasonic scan line.

Specifically, with the Doppler processing function 182, the processing circuitry 180 estimates, at each sampling point, an average velocity, an average dispersion value, an average power value, etc., for example, as motion information of a moving object, and generates Doppler data showing the estimated motion information. The moving object is a bloodstream, tissue such as the cardiac wall, a contrast agent, etc. With the Doppler processing function 182, the processing circuitry 180 according to the first embodiment estimates, at each sampling point, an average blood flow rate, a dispersion value of a blood flow rate, a power value of a blood flow signal, etc., as motion information of bloodstream (blood flow information), and generates Doppler data showing the estimated blood flow information.

Also, with the Doppler processing function 182, the processing circuitry 180 can perform a color Doppler method also referred to as a color flow mapping (CFM) method. In the CFM method, transmission and reception of ultrasonic waves on multiple scan lines are performed multiple times. In the CFM method, a moving target indicator (MTI) filter is applied to data columns in the same position, for example, whereby signals (clutter signals) originating from static tissue or slow-moving tissue are suppressed and signals originating from a blood flow are extracted. In the CFM method, the extracted blood flow signals are used to estimate blood flow information such as blood flow rate, blood flow dispersion, and blood flow power. With the image generating function 183 described later, a distribution of the estimated blood flow information is generated, for example, as ultrasonic image data (color Doppler image data) that is displayed two-dimensionally in color. Hereinafter, the mode of the ultrasonic diagnostic apparatus that adopts the color Doppler method will be referred to as a “blood flow imaging mode”. Color display refers to displaying a distribution of the blood flow information in accordance with a predetermined color code, and includes gray-scale color display.

There are various types of blood flow imaging modes depending on desired clinical information. In general, there is a blood flow imaging mode for displaying velocity that allows for visualization of a blood flow direction or an average blood flow rate, and a blood flow imaging mode for displaying power that allows for visualization of blood flow signal power.

The blood flow imaging mode for displaying velocity is a mode of displaying color corresponding to the Doppler shift frequency based on a blood flow direction or an average blood flow rate. For example, the blood flow imaging mode for displaying velocity represents, as flow directions, an approaching flow by a red-based color and a receding flow by a blue-based color, thereby representing the difference in the velocity between the approaching flow and the receding flow by the difference in the hue. The blood flow imaging mode for displaying velocity may also be referred to as a “color Doppler mode” or a “color Doppler imaging (CDI) mode”.

The blood flow imaging mode for displaying power is a mode of representing blood flow signal power, for example, by a red-based hue, brightness of the color, or a change in chromaticness. The blood flow imaging mode for displaying power may also be referred to as a “power Doppler (PD) mode”. Since the blood flow imaging mode for displaying power can represent the blood flow at a high sensitivity, as compared to the blood flow imaging mode for displaying velocity, the blood flow imaging mode for displaying power may be referred to as a high-sensitivity blood flow imaging mode.

The image generating function 183 is a function to generate B-mode image data based on the data generated by the B-mode processing function 181. With the image generating function 183, the processing circuitry 180, for example, converts (scan-converts) a scan line signal sequence of ultrasonic scanning into a scan line signal sequence in a video format representatively used by a television, etc., and generates image data for display (display image data). Specifically, the processing circuitry 180 generates two-dimensional B-mode image data (also referred to as “ultrasonic image data”) constituted by pixels by executing RAW-pixel conversion, such as coordinate conversion according to the mode of the ultrasonic scanning performed by the ultrasonic probe 101, on B-mode RAW data stored in the RAW data memory. In other words, with the image generating function 183, the processing circuitry 180 generates multiple ultrasonic images (medical images) corresponding to respective consecutive frames through ultrasonic transmission and reception.

The image generating function 183 also has a function to generate Doppler image data based on the data generated by the Doppler processing function 182. For example, the image generating function 183 generates Doppler image data of visualized blood flow information by executing RAW-pixel conversion on the Doppler RAW data stored in the RAW data memory. The Doppler image data is average velocity image data, dispersion image data, power image data, or combined image data thereof. The processing circuitry 180 generates, as the Doppler image data, color Doppler image data showing colored blood flow information and Doppler image data showing a piece of blood flow information in waveform on a gray scale. The color Doppler image data is generated when the above-described blood flow imaging modes are executed.

The setting function 184 sets a scan order such that, among multiple ensemble groups, a distance between temporally adjacent ultrasonic transmission-reception operations of different ensemble groups is not less than a threshold, and a time difference between ultrasonic transmission-reception operations of the different ensemble groups with respect to spatially adjacent scan lines is not less than a threshold. The ensemble groups each include multiple ultrasonic transmission-reception operations performed on the same scan line in each of partial scan regions that are partial areas of a scan region.

The transmission-reception control function 185 causes ultrasonic waves to be transmitted via the ultrasonic transmitter circuitry 110 according to the scan order set by the setting function 184 and causes echo signals from the living body P to be received via the ultrasonic receiver circuitry 120, thereby generating received signals that are aligned in the scan order.

The display control function 186 is a function to cause a display as the output device 103 to display images based on various kinds of ultrasonic image data generated by the image generating function 183. Specifically, with the display control function 186, the processing circuitry 180, for example, controls the display of an image that is based on the B-mode image data, the Doppler image data, or image data including both that is generated by the image generating function 183.

More specifically, with the display control function 186, the processing circuitry 180, for example, converts (scan-converts) a scan line signal sequence of ultrasonic scanning into a scan line signal sequence in a video format representatively used by a television, etc., and generates display image data. The processing circuitry 180 may also perform various kinds of processing, such as corrections of dynamic range, brightness, contrast, and a γ-curve and RGB conversion, on the display image data. The processing circuitry 180 may also add supplementary information, such as textual information of various parameters, a scale, or a body mark, to the display image data. The processing circuitry 180 may also generate a user interface (graphical user interface: GUI) for an operator to input various commands through the input device, and cause the display to display the GUI.

The system control function 187 is a function to perform overall control of the operations of the ultrasonic diagnostic apparatus 1. For example, with the system control function 187, the processing circuitry 180 controls the ultrasonic transmitter circuitry 110 and the ultrasonic receiver circuitry 120 so as to execute scanning for transmit aperture synthesis during execution of an examination mode (contrast examination mode) using a contrast agent.

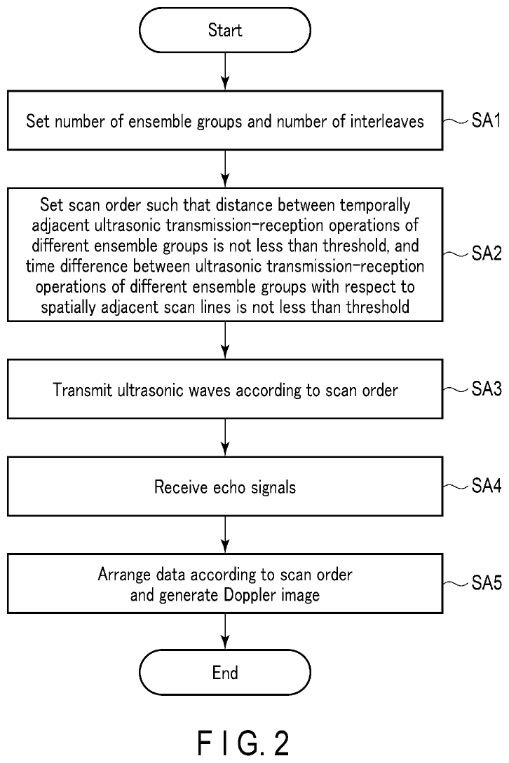

Next, an example of an operation of the ultrasonic diagnostic apparatus according to the embodiment will be described with reference to the flowchart of FIG. 2.

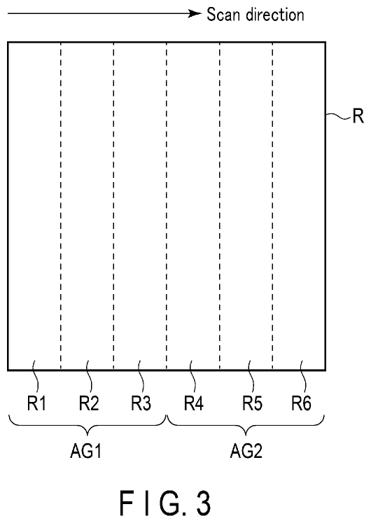

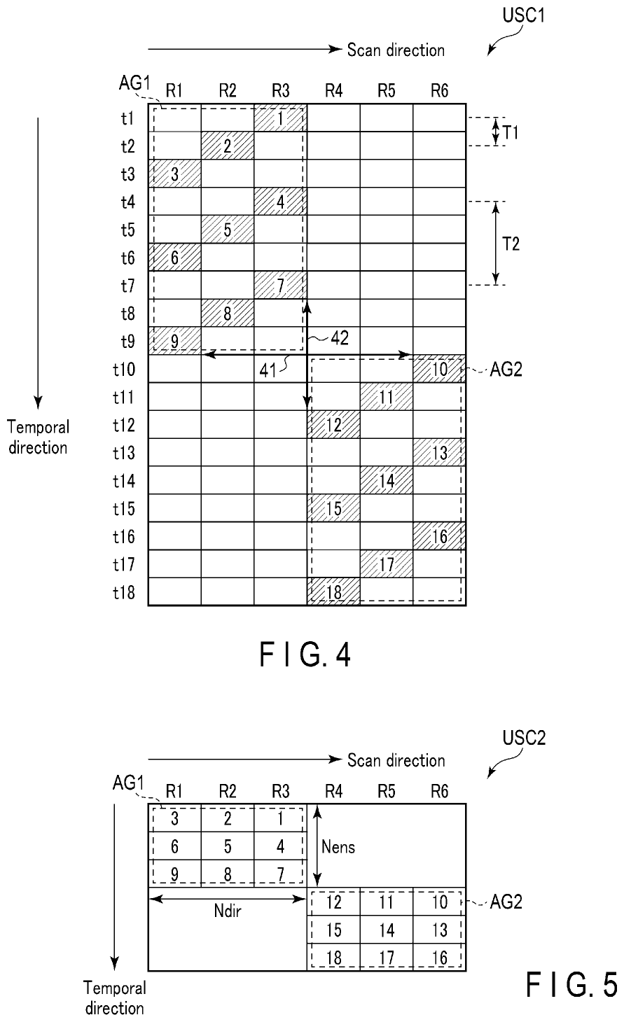

In step SA1, the processing circuitry 180 implements the setting function 184 to determine the number of ensemble groups and the number of interleaves. The number of ensemble groups is the number of ensemble groups set in a scan region. For example, if the scan region is divided into three partial regions, the number of ensemble groups is “3”. The number of interleaves is the number of transmission beams, that is, the number of scan lines, included in a single ensemble group. For example, if four scan lines are included in an ensemble group, the number of interleaves is “4”. Detailed descriptions thereof will be given later with reference to FIGS. 3 and 4.

In step SA2, with the setting function 184, the processing circuitry 180 sets a scan order such that, among multiple ensemble groups each including multiple ultrasonic transmission-reception operations performed on the same scan line, a distance between temporally adjacent ultrasonic transmission-reception operations of different ensemble groups is not less than a threshold, and a time difference between ultrasonic transmission-reception operations of the different ensemble groups with respect to spatially adjacent scan lines is not less than a threshold.

For example, with the setting function 184, the processing circuitry 180 sets a scan order relating to ultrasonic transmission-reception operations between ensemble groups and within an ensemble group such that at least one of a distance or a time difference between the last ultrasonic transmission-reception operation in a first ensemble group and the first ultrasonic transmission-reception operation in a second ensemble group on which scanning is performed after the first ensemble group is not less than a threshold.

In step SA3, with the transmission-reception control function 185, the processing circuitry 180 transmits ultrasonic waves from the ultrasonic probe 101 to the living body P via the ultrasonic transmitter circuitry 110 according to the scan order set in step SA2.

In step SA4, with the transmission-reception control function 185, the processing circuitry 180 receives echo signals reflected inside the living body P via the ultrasonic receiver circuitry 120.

In step SA5, with the transmission-reception control function 185, the processing circuitry 180 rearranges the echo signals according to the scan order, and with the image generating function 183, the processing circuitry 180 generates a Doppler image. The generated Doppler image is displayed on the output device 103 such as a display.

Next, a scan region according to the embodiment will be described with reference to FIG. 3.

FIG. 3 illustrates a region R of interest from which color Doppler image data is collected by the ultrasonic probe 101. For convenience of explanation, an entire scan region is defined as the region R of interest of a color Doppler image.

As shown in FIG. 3, the ultrasonic diagnostic apparatus 1 performs a color Doppler-mode scan of the region R of interest, for example, in order to collect color Doppler image data corresponding to one frame. This region R of interest is constituted by, for example, six beams (scan lines) that are transmitted and received. Specifically, the ultrasonic diagnostic apparatus 1 divides the region R of interest into a region R1 to a region R6 respectively corresponding to the six beams.

In addition, the ultrasonic diagnostic apparatus 1 performs scanning by dividing the scan region as the region R of interest into multiple groups so as to have multiple partial scan regions. Specifically, the ultrasonic diagnostic apparatus 1 scans the region R of interest by dividing it into two ensemble groups, an ensemble group AG1 including the region R1 to the region R3, and an ensemble group AG2 including the region R4 to the region R6. Thus, each ensemble group is constituted by three beams.

Next, a first example of setting a scan order with the setting function 184 according to the embodiment will be described with reference to FIG. 4.

FIG. 4 is an ultrasonic scan chart USC1 showing the order of ultrasonic transmission-reception. The horizontal direction of the ultrasonic scan chart USC1 is a scan direction, and corresponds to the ultrasonic transmission-reception operations in the region R1 to the region R6 shown in FIG. 3. The vertical direction of the ultrasonic scan chart USC1 is a temporal direction, and corresponds to the order of the ultrasonic transmission-reception operations in the region R1 to the region R6. The number indicated in the ultrasonic scan chart USC1 corresponds to the time of ultrasonic transmission-reception.

In the CFM method, a data column of reflected wave data in the same position is used to generate blood flow information corresponding to one frame. Thus, the ultrasonic diagnostic apparatus 1 collects data columns in respective positions (sample points) in the region R of interest by repeatedly performing a color Doppler-mode scan of the region R of interest. For example, the ultrasonic diagnostic apparatus 1 collects color Doppler image data corresponding to one frame by performing a color Doppler-mode scan of the region R of interest three times in a predetermined repetition cycle. In the example shown in FIG. 4, the ultrasonic diagnostic apparatus 1 performs a color Doppler-mode scan in each of the ensemble group AG1 and the ensemble group AG2 three times for each different time period. In other words, the repetition cycle corresponds to the cycle of repeating the color Doppler-mode scan. Note that the number of times of repeating the color Doppler-mode scan in a group is called the number of ensembles Nens. The number of ensembles Nens is specified by a user.

Herein, acoustic pulse repetition frequency (PRF) corresponds to an inverse of a period (time period) from transmission of a beam to transmission of the next beam. That is, since an inverse “f-Inv” of the acoustic PRF, for example, corresponds to the time period from execution of ultrasonic transmission and reception to execution of the next ultrasonic transmission and reception, it can be said to correspond to a transmission-reception time T1 required to transmit and receive each beam. The acoustic PRF is determined based on, for example, at least one of a position of a lower end (depth) of the region R of interest, a flow rate range, or a reception frequency of ultrasonic waves. In other words, the ultrasonic diagnostic apparatus 1 determines the transmission-reception time T1 based on at least one of a depth of the region R of interest (a depth of ROI), a flow rate range, or a reception frequency of ultrasonic waves. In the example shown in FIG. 4, the transmission-reception time T1 corresponds to, for example, a time period from execution of ultrasonic transmission and reception in the region R3 at the time t1 to execution of ultrasonic transmission and reception in the region R2 at the time t2.

Next, the ultrasonic diagnostic apparatus 1 calculates a repetition cycle T2 of a color Doppler-mode scan. The repetition cycle T2 corresponds to a period (time period) in which transmission and reception in a region are repeatedly performed. That is, the repetition cycle T2 corresponds to, for example, a time period from execution of ultrasonic transmission and reception in a region to re-execution of ultrasonic transmission and reception in the same region through a period of ultrasonic transmission and reception in another region for multiple-time performance of a color Doppler-mode scan in a group. If a detected maximum flow rate is a high flow rate, the repetition cycle T2 is reduced since the next ultrasonic transmission and reception need to be performed early, whereas the repetition cycle T2 is increased if a detected maximum flow rate is a low flow rate. Thus, the ultrasonic diagnostic apparatus 1 calculates the repetition cycle T2 based on a detected maximum flow rate of a set flow rate range. The repetition cycle T2 has the same value in each region created by dividing the region of interest. In the example shown in FIG. 4, the repetition cycle T2 corresponds to, for example, a time period from execution of ultrasonic transmission and reception in the region R3 at the time t4 to re-execution of ultrasonic transmission and reception in the region R3 at the time t7.

Next, the ultrasonic diagnostic apparatus 1 calculates the number of interleaves (the number of transmission directions in alternate stages) Ndir of interleave scanning based on the repetition cycle T2 and the acoustic PRF (or the transmission-reception time T1). The interleave scanning referred to herein is a method that does not continuously perform ultrasonic transmission-reception operations in one region when collecting data columns in a predetermined region (e.g., one region) by the CFM method, but groups multiple regions as one group and sequentially performs ultrasonic transmission-reception operations in the multiple regions included in the group. The example shown in FIG. 4 is that, in the ensemble group of the three regions, the region R1 to the region R3, after ultrasonic waves are sequentially transmitted to the region R1 to the region R3, echo signals are sequentially received from the region R1 to the region R3, and this process is repeated a predetermined number of times. In this interleave scanning, the number of regions included in one ensemble group is referred to as “the number of interleaves Ndir”. That is, the number of interleaves Ndir corresponds to the number of regions included in each group, in other words, the number of scan lines.

The example illustrated in FIG. 4 shows the case where both the number of ensembles Nens and the number of interleaves Ndir are set to “3”.

In the first example of setting a scan order according to the embodiment, the processing circuitry 180 implements the setting function 184 to set a scan order such that the scan direction of each ensemble group is opposite to the scan direction of the entire scan region. Specifically, whereas the scan direction of the ultrasonic scan chart USC1 corresponding to the scan region is from the region R1 to the region R6, scanning is performed in the ensemble group AG1 in a direction from the region R3 to the region R1, as in an ultrasonic transmission-reception operation in the region R3 performed at the time t1, an ultrasonic transmission-reception operation in the region R2 performed at the time t2, and an ultrasonic transmission-reception operation in the region R1 performed at the time t3. Likewise, in the ensemble group AG2, scanning is performed in a direction from the region R6 to the region R4, as in an ultrasonic transmission-reception operation in the region R6 performed at the time t10, an ultrasonic transmission-reception operation in the region R5 performed at the time t11, and an ultrasonic transmission-reception operation in the region R4 performed at the time t12.

First, the ultrasonic diagnostic apparatus 1 performs a color Doppler-mode scan in the ensemble group AG1 three times for each different time period. Specifically, the ultrasonic diagnostic apparatus 1 performs ultrasonic transmission-reception operations corresponding to the regions R3 to R1 from the time t1 to the time t3, performs ultrasonic transmission-reception operations corresponding to the regions R3 to R1 from the time t4 to the time t6, and performs ultrasonic transmission-reception operations corresponding to the regions R3 to R1 from the time t7 to the time t9.

After performing scanning in the ensemble group AG1, the ultrasonic diagnostic apparatus 1 performs a color Doppler-mode scan in the ensemble group AG2 three times for each different time period. Specifically, the ultrasonic diagnostic apparatus 1 performs ultrasonic transmission-reception operations corresponding to the regions R6 to R4 from the time t10 to the time t12, performs ultrasonic transmission-reception operations corresponding to the regions R6 to R4 from the time t13 to the time t15, and performs ultrasonic transmission-reception operations corresponding to the regions R6 to R4 from the time t16 to the time t18.

After performing scanning in the ensemble group AG2, the ultrasonic diagnostic apparatus 1 alternately performs color Doppler-mode scan operations in each of the ensemble group AG1 and the ensemble group AG2 in the same manner from then onward.

If the scan direction of the scan region and the scan direction of each ensemble group is made to be the same, as in the conventional method, the last ultrasonic transmission-reception operation in the ensemble group AG1 is performed at the time t9 in the region R3, and the first ultrasonic transmission reception operation in the ensemble group AG2, which is the next target to be scanned, is performed at the time t10 in the region R4. Thus, a distance between temporally adjacent ultrasonic transmission-reception operations of different ensemble groups, that is, a distance between the regions where ultrasonic transmission-reception operations are performed at the time t9 and time t10, is “1”. A time difference between ultrasonic transmission-reception operations in the spatially adjacent regions of the different ensemble groups, that is, a time difference in which ultrasonic transmission-reception operations are performed in the region R3 and the region R4, is also “1”.

On the other hand, according to the scan order of the first setting example of the embodiment shown in FIG. 4, a distance 41 between temporally adjacent ultrasonic transmission-reception operations of different ensemble groups, that is, a distance 41 between the time t9 at which the last ultrasonic transmission-reception operation is performed in the ensemble group AG1 and the time t10 at which the first ultrasonic transmission-reception operation is performed in the ensemble group AG2, which is the next target to be scanned, can be set to a distance difference corresponding to “5” regions, which is a difference between the region R1 and the region R6.

In addition, in the region R3 and the region R4 adjacent to each other and forming a boundary between the ensemble group AG1 and the ensemble group AG2, a time difference 42 between the time t7 at which an ultrasonic transmission-reception operation is performed in the region R3 and the time t12 at which an ultrasonic transmission-reception operation is performed in the region R4 can be set to a time difference corresponding to “5” transmission-reception times T1.

Thus, a distance interval can be provided for temporally adjacent ultrasonic transmission-reception operations of different ensemble groups (the time t9 and the time t10 in FIG. 4), and a time interval of ultrasonic transmission-reception operations can be provided for the spatially adjacent regions (the region R3 and the region R4 in FIG. 4). As a result, residual multiplex noise that affects imaging can be greatly reduced, as compare to the conventional method.

Next, a simplified version of the ultrasonic scan chart shown in FIG. 4 will be described with reference to FIG. 5.

The content of the ultrasonic scan chart USC2 shown in FIG. 5 is the same as the content of the ultrasonic scan chart USC1 shown in FIG. 4. Specifically, in the ultrasonic scan chart USC2, illustration of the temporal direction of the ultrasonic scan chart USC1 is compressed. For example, in the ultrasonic scan chart USC2, the lateral direction corresponds to a set of the number of interleaves Ndir. Thus, the first line of the ultrasonic scan chart USC2 corresponds to the three lines from the time t1 to the time t3 of the ultrasonic scan chart USC1. Accordingly, the order of the ultrasonic transmission-reception operations illustrated in eighteen lines in the ultrasonic scan chart USC1 can be illustrated in six lines in the ultrasonic scan chart USC2. Hereinafter, illustration of the ultrasonic scan chart USC2 will be used for explanation.

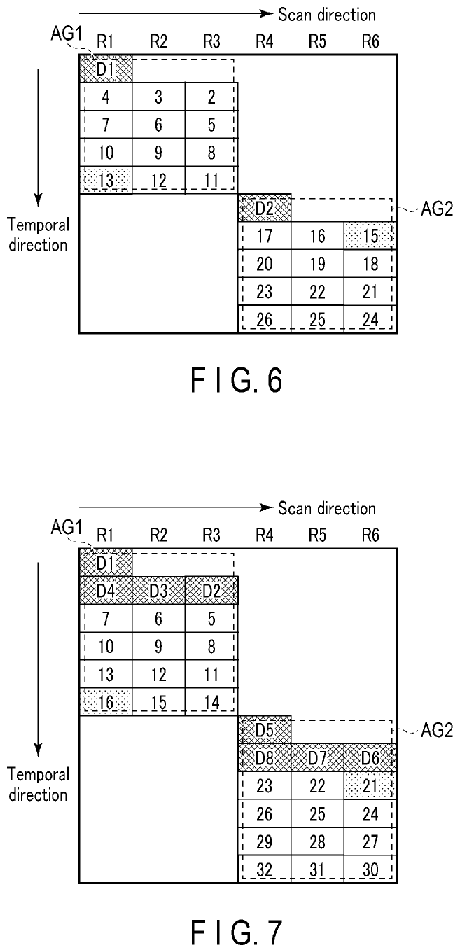

Next, a second example of setting a scan order with the setting function 184 according to the embodiment will be described with reference to FIG. 6.

FIG. 6 shows an example of setting dummy ultrasonic transmission-reception in which echo signals obtained through ultrasonic transmission and reception are not used for synthesizing a Doppler image. Let us assume a case where the number of interleaves Ndir is set to “3” and the number of ensembles Nens is set to “4” in each of the ensemble group AG1 and the ensemble group AG2.

The first ultrasonic transmission-reception operation in the ensemble group AG1, that is, the ultrasonic transmission-reception operation in the region R1 at the time t1, is set to a dummy ultrasonic transmission-reception operation D1, and, as in the case shown in FIG. 5, a scan order of ultrasonic transmission-reception operations is set from the ultrasonic transmission-reception operation in the region R3 at the time t2 in a direction opposite to the scan direction of the scan region. Likewise, the first ultrasonic transmission-reception operation in the ensemble group AG2, that is, the ultrasonic transmission-reception operation in the region R4 at the time t14, is set to a dummy ultrasonic transmission-reception operation D2, and a scan order of ultrasonic transmission-reception operations is set from the ultrasonic transmission-reception operation in the region R6 at the time t15 in a direction opposite to the scan direction of the scan region.

Thus, the time t14 following the last ultrasonic transmission-reception operation in the ensemble group AG1 (the region R1 at the time t13) is treated as dummy data, allowing a greater time difference to be secured until the first ultrasonic transmission-reception operation in the ensemble group AG2 (the region R6 at the time t15) is performed, as compared to the case shown in FIG. 4. Accordingly, residual multiplex noise can be reduced to a greater degree, as compared to the first example of setting a scan order.

Next, a third example of setting a scan order with the setting function 184 according to the embodiment will be described with reference to FIG. 7.

While FIG. 6 shows an example of performing a single dummy ultrasonic transmission-reception operation in each ensemble group, the example shown in FIG. 7 differs therefrom in that multiple dummy ultrasonic transmission-reception operations are performed in each ensemble group. Four dummy ultrasonic transmission-reception operations D1 to D4 are performed from the beginning of the ensemble group AG1. Specifically, a dummy ultrasonic transmission-reception operation D1 is performed on the region R1 at the time t1, a dummy ultrasonic transmission-reception operation D2 is performed on the region R3 at the time t2, a dummy ultrasonic transmission-reception operation D3 is performed on the region R2 at the time t3, and a dummy ultrasonic transmission-reception operation D4 is performed on the region R1 at the time t4. Likewise, in the ensemble group AG2, a dummy ultrasonic transmission-reception operation D5 is performed on the region R4 at the time t17, a dummy ultrasonic transmission-reception operation D6 is performed on the region R6 at the time t18, a dummy ultrasonic transmission-reception operation D7 is performed on the region R5 at the time t19, and a dummy ultrasonic transmission-reception operation D8 is performed on the region R4 at the time t20.

In this manner, according to the third example of setting a scan order shown in FIG. 7, the time t17 following the last ultrasonic transmission-reception operation in the ensemble group AG1 (the region R1 at the time t16) to the time t20 are dummy data, allowing a greater time difference to be secured until the ultrasonic transmission-reception operation at the head of the ensemble group AG2 (the region R6 at the time t21) is performed, as compared to the case shown in FIG. 6. Accordingly, residual multiplex noise can be reduced to a greater degree, as compared to the second example of setting a scan order.

While an increased number of dummy ultrasonic transmission-reception operations can reduce residual multiplex noise, it is in a trade-off relation with a frame rate in that an increased number of dummy ultrasonic transmission-reception operations deceases the frame rate. To address this issue, the processing circuitry 180 may implement the setting function 184 to set the number of dummy ultrasonic transmission-reception operations according to the acceptable frame rate.

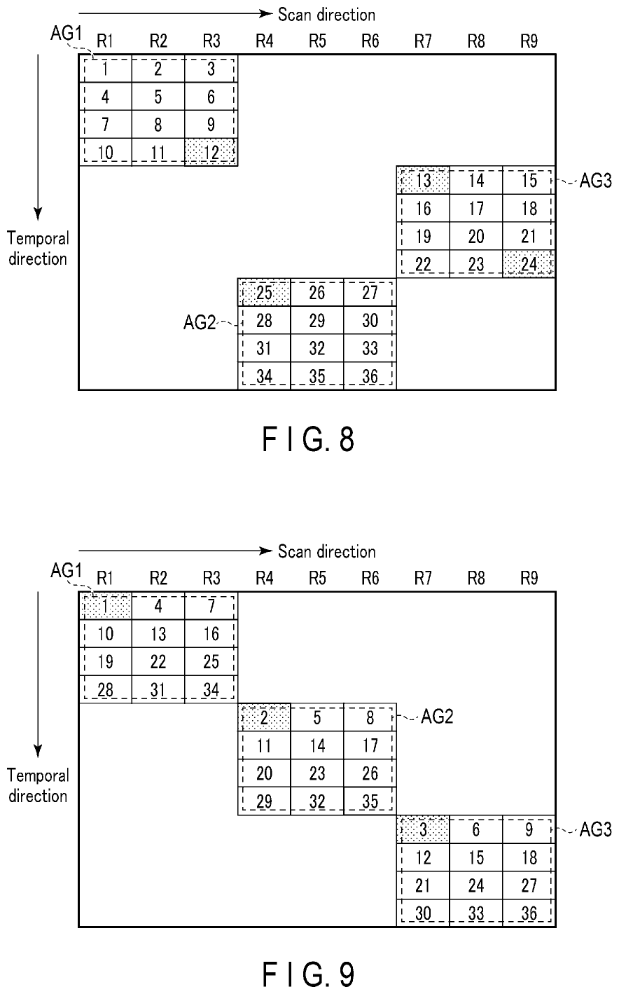

Next, a fourth example of setting a scan order according to the embodiment will be described with reference to FIG. 8.

In the example described above, a scan order is set such that the scan direction of the scan region is opposite to the scan direction within the ensemble group. On the other hand, FIG. 8 shows an example in which the scan direction of the scan region is the same as the scan direction within each ensemble group. In this case, the processing circuitry 180 implements the setting function 184 to set a scan order such that adjacent ensemble groups are not scanned continuously but at least one adjacent ensemble group is skipped with respect to the scan direction of the ensemble group.

Specifically, after ultrasonic transmission-reception operations are performed in the ensemble group AG1 in a scan order in the same direction as the scan direction of the scan region, the ensemble group AG2 is skipped, and ultrasonic transmission-reception operations are performed in the ensemble group AG3 in a scan order in the same direction as the scan direction of the scan region. Thereafter, the remaining ensemble group AG2 may be scanned.

This allows for spacing between the last ultrasonic transmission-reception operation in the ensemble group AG1 (the region R3 at the time t12) and the first ultrasonic transmission-reception operation in the ensemble group AG3 (the region R7 at the time t13) by the distance corresponding to “4” regions. Subsequently, spacing can be provided between the last ultrasonic transmission-reception operation in the ensemble group AG3 (the region R9 at the time t24) and the first ultrasonic transmission-reception operation in the ensemble group AG2 (the region R4 at the time t25) by the distance corresponding to “5” regions.

Also, a time difference corresponding to “13” transmission-reception times T1 can be secured between spatially adjacent regions of different ensemble groups since the region R3 of the ensemble group AG1 has the ultrasonic transmission-reception operation at the time t12, and the region R4 of the ensemble group AG2 has the ultrasonic transmission-reception operation at the time t25. A time difference corresponding to “5” transmission-reception times T1 can be secured since the region R6 of the ensemble group AG2 has the ultrasonic transmission-reception operation at the time t27, and the region R7 of the ensemble group AG3 has the ultrasonic transmission-reception operation at the time t22.

Although the example shown in FIG. 8 describes the scan order of the three ensemble groups AG1 to AG3, the processing circuitry 180 may also implement the setting function 184 to set a scan order for four or more ensemble groups such that an adjacent ensemble group is skipped. For example, if there are five ensemble groups AG1 to AG5 along the scan direction of the scan region, a scan order may be set in the order of the ensemble group AG1, the ensemble group AG3, the ensemble group AG5, the ensemble group AG2, and the ensemble group AG4 so that adjacent ensemble groups are skipped.

Next, a fifth example of setting a scan order according to the embodiment will be described with reference to FIG. 9.

The fifth setting example is an example of setting a scan order so as to perform ultrasonic transmission-reception operations by selecting regions belonging to the respective ensemble groups one by one. Specifically, an ultrasonic transmission-reception operation is performed on the region R1 of the ensemble group AG1 at the time t1, an ultrasonic transmission-reception operation is performed on the region R4 of the ensemble group AG2 at the time t2, and an ultrasonic transmission-reception operation is performed on the region R7 of the ensemble group AG3 at the time t3. Thereafter, returning to the ensemble group AG1, an ultrasonic transmission-reception operation is performed on the region R2 at the time t4. In this manner, a scan order is set such that regions where ultrasonic transmission-reception operations are performed are selected one by one among the ensemble groups AG1 to AG3.

Thus, a distance between the region R1 (the time t1) and the region R4 (the time t2) and a distance between the region R4 (the time t2) and the region R7 (the time t3), that is, a distance corresponding to “3” regions, can be secured for the temporally adjacent ultrasonic transmission-reception operations of different ensemble groups.

A time difference between the time t7 (the region R3) and the time t2 (the region R4) and a time difference between the time t8 (the region R6) and the time t3 (the region R7), that is, a time difference corresponding to “5” transmission-reception times T1, can also be secured for the spatially adjacent regions of different ensemble groups.

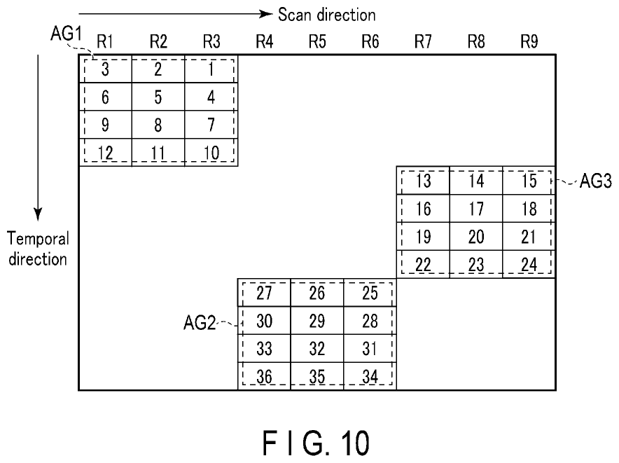

Next, a sixth example of setting a scan order according to the embodiment will be described with reference to FIG. 10.

The example shown in FIG. 10 is an instance of combining the above-described setting examples, and a combination of the first example of setting a scan order and the fourth example of setting a scan order is shown herein. Specifically, after ultrasonic transmission-reception operations are performed from the time t1 to the time t12 in the ensemble group AG1 in a scan order in a direction opposite to the scan direction of the scan region, the ensemble group AG2 adjacent to the ensemble group AG1 is skipped, and ultrasonic transmission-reception operations are performed from the time t13 to the time t24 in the ensemble group AG3 in a scan order in the same direction as the scan direction of the scan region. Thereafter, ultrasonic transmission-reception operations are performed from the time t25 to the time t36 in the ensemble group AG2 in a scan order in a direction opposite to the scan direction of the scan region.

Thus, a distance corresponding to “6” regions, which is a distance between the region R1 (the time t12) of the ensemble group AG1 and the region R7 (the time t13) of the ensemble group AG3, and a distance corresponding to “3” regions, which is a distance between the region R9 (the time t24) of the ensemble group AG3 and the region R6 (the time t25) of the ensemble group AG2, can be secured for the temporally adjacent ultrasonic transmission-reception operations of different ensemble groups.

A time difference corresponding to “17” transmission-reception times T1, which is a time difference between the time t10 (the region R3) and the time t27 (the region R4), can also be secured for the spatially adjacent regions of different ensemble groups. A time difference corresponding to “3” transmission-reception times T1, which is a time difference between the time t22 (the region R7) and the time t25 (the region R6), can also be secured.

While the respective examples of setting a scan order can be combined, as described above, the criterion for the combination may be such that a distance difference between the regions relating to ultrasonic transmission-reception is not less than a threshold for the temporally adjacent ultrasonic transmission-reception operations of different ensemble groups. This threshold may be set based on actual measurement data, for example, such that residual multiplex noise is not generated or an influence of residual multiplex noise is at an acceptable level, in other words, such that residual multiplex noise is not more than a predetermined value. Also, a time difference relating to ultrasonic transmission-reception may be set to not less than a threshold in the spatially adjacent regions of different ensemble groups. Likewise, this threshold may also be set based on actual measurement data, etc., such that residual multiplex noise is not more than a predetermined value.

According to the embodiment described above, the processing circuitry implements the setting function to set a scan order relating to ultrasonic transmission-reception such that a distance difference between temporally adjacent ultrasonic transmission-reception operations of different ensemble groups is not less than a threshold and that a time difference between spatially adjacent regions of different ensemble groups is not less than a threshold. Thus, it is possible to greatly reduce an influence of residual multiplex noise on the ultrasonic transmission-reception operations. As a result, it is possible to reduce noise while maintaining the frame rate.

According to at least one embodiment described above, it is possible to reduce noise while maintaining the frame rate.

The term “processor” used in the above description means, for example, circuitry such as a central processing unit (CPU), a graphics processing unit (GPU), an application-specific integrated circuit (ASIC), or a programmable logic device (e.g., a simple programmable logic device (SPLD), a complex programmable logic device (CPLD), or a field-programmable gate (FPGA)). If the processor is, for example, a CPU, the processor implements the functions by reading and executing programs stored in storage circuitry. On the other hand, if the processor is an ASIC, for example, its functions are directly incorporated into the circuitry of the processor as logic circuitry, instead of a program being stored in the storage circuitry. Each processor of the embodiment is not limited to a single circuitry-type processor, and multiple independent circuits may be combined and integrated as a single processor to implement the intended functions. Furthermore, the functions may be implemented by a single processor into which multiple components shown in the drawings are incorporated.

In addition, the functions described in the above embodiment may be implemented by installing programs for executing the processing in a computer, such as a workstation, and expanding the programs in a memory. The programs that can cause the computer to execute the processing can be stored in a storage medium, such as a magnetic disk (a hard disk, etc.), an optical disk (CD-ROM, DVD, etc.), or a semiconductor memory, and distributed through it.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising processing circuitry configured to:
set a scan order such that among multiple ensemble groups each including multiple ultrasonic transmission-reception operations performed on a same scan line in each of partial scan regions of a scan region, a distance between temporally adjacent ultrasonic transmission-reception operations of different ensemble groups is not less than a first threshold, and a time difference between ultrasonic transmission-reception operations of the different ensemble groups with respect to spatially adjacent scan lines is not less than a second threshold; and
cause ultrasonic waves to be transmitted and received according to the scan order.

2. The ultrasonic diagnostic apparatus according to claim 1, wherein the processing circuitry sets the scan order such that a scan direction in each ensemble group is opposite to a scan direction of the scan region.

3. The ultrasonic diagnostic apparatus according to claim 1, wherein the processing circuitry sets the scan order such that a distance between a last ultrasonic transmission-reception operation in a first ensemble group and a first ultrasonic transmission-reception operation in a second ensemble group on which scanning is performed after the first ensemble group is not less than the first threshold.

4. The ultrasonic diagnostic apparatus according to claim 1, wherein the processing circuitry sets one or more ultrasonic transmission-reception operations from a head in each ensemble group as dummy ultrasonic transmission-reception that is not used for synthesizing a Doppler image.

5. The ultrasonic diagnostic apparatus according to claim 1, wherein the processing circuitry sets the scan order such that at least one adjacent ensemble group is skipped with respect to a direction of scanning multiple ensemble groups in the scan region.

6. The ultrasonic diagnostic apparatus according to claim 1, wherein the processing circuitry sets the scan order such that ultrasonic transmission-reception operations of each ensemble group are sequentially performed one by one.

7. The ultrasonic diagnostic apparatus according to claim 1, wherein the processing circuitry is further configured to generate a Doppler image based on an echo signal received according to the scan order.

8. The ultrasonic diagnostic apparatus according to claim 1, wherein the first threshold and the second threshold are set based on a distance and a time that makes residual multiplex noise not more than a predetermined value.

9. A non-transitory computer readable medium including computer executable instructions, wherein the instructions, when executed by a processor, cause the processor to perform a method comprising:
setting a scan order such that among multiple ensemble groups each including multiple ultrasonic transmission-reception operations performed on a same scan line in each of partial scan regions of a scan region, a distance between temporally adjacent ultrasonic transmission-reception operations of different ensemble groups is not less than a first threshold, and a time difference between ultrasonic transmission-reception operations of the different ensemble groups with respect to spatially adjacent scan lines is not less than a second threshold; and
causing ultrasonic waves to be transmitted and received according to the scan order.
